(19) United States
(12) Patent Application Publication
(10) Pub. No.: US 2014/0171945 A1
(43) Pub. Date: Jun. 19, 2014

(54) APPARATUS AND METHOD FOR BONE FIXATION

(71) Applicant: Bill Reese, Gilbert, AZ (US)

(72) Inventor: Bill Reese, Gilbert, AZ (US)

(21) Appl. No.: 13/720,629

(22) Filed: Dec. 19, 2012

(51) Int. Cl. A61B 17/82

(52) U.S. Cl. CPC A61B 17/82
USPC 606/74

(57) ABSTRACT

The various implementations of the present invention provide an improved apparatus for bone reduction and fixation. A flat, substantially rectangular band comprises a wing portion and a plurality of apertures formed in the body of the band. The band provides a mechanism for fixing bones in a desired position by encircling a first bone and a second bone. A first set of apertures provides for real time adjustment of the length of the band to allow for a wide variety of bone reduction applications. By selecting the appropriate length for the band and inserting the wing portion in the appropriate slot, the band can be used for a wide range of applications. A second set of apertures formed in the band provide for bone and tissue contact, promoting healing of the body where the band is placed. A method for determining the proper positioning and placement of the wing portion during the bone reduction process is also disclosed.

### BACKGROUND OF THE INVENTION

1. Technical Field

The present invention relates generally to the field of orthopedic surgery and more specifically relates to fixation of bones for orthopedic procedures.

2. Background Art

The human foot is an intricate structure comprising 24 bones that form two crossing arches of the foot. The longitudinal arch runs the length of the foot, and the transverse arch runs the width. The bones of the foot are primarily held together by their fit with each other and are connected by a fibrous tissue known as ligaments. The muscles of the foot, along with a tough, sinewy tissue known as the plantar fascia, provide secondary support to the foot. While generally functional, there can be instances where the structure of the foot is compromised. Two of the most common foot ailments are broken bone and bunions.

Broken bones, also called fractures, are very common in the human foot. The feet are generally vulnerable to slipping and twisting during normal activities and even more so during sporting or athletic events. For example, prolonged repetitive movements, experienced during certain sports activities, can cause a type of broken type of broken toe called a “stress” fracture or “hairline” fracture. Additionally, 1 the feet and toes are often on the receiving end of dropped objects. Statistics indicated that about one out of every 10 broken bones occurs in the foot. Once fractured, it may be necessary to perform orthopedic surgery to reposition one or more fractured bones for proper healing.

Bunions, another common foot ailment, are generally characterized by a localized area of enlargement of the inner portion of the joint at the base of the big toe. The enlargement represents additional bone formation, often in combination with a misalignment of the big toe. The misalignment may cause the big toe to move outward and the enlarged joint at the base of the big toe can become inflamed with redness, tenderness, and pain. A small fluid-filled sac adjacent to the toe bone joint can also become inflamed, leading to additional swelling, redness, and pain. A less common bunion is located at the joint at the base of the smallest toe. This bunion is sometimes referred to as a tailor's bunion and may also cause bone misalignment.

To remedy the problems associated with fractures or misaligned bones due to bunions, orthopedic intervention is often necessary. For example, an “osteotomy” is a surgical operation whereby a bone is cut to shorten, lengthen, or change its alignment. It is sometimes performed to correct a deformity, or to straighten a bone that has healed crookedly following a fracture. Similarly, “reduction” is an orthopedic procedure to restore a fractured or dislocated bone to the correct alignment. When a bone fractures, the fragments lose their alignment in the form of displacement or angulation. For the fractured bone to heal without any deformity the bony fragments must be re-aligned to their normal anatomical position.

Many types of devices are readily available both for fracture fixation and for the fixation of bones that are to be repositioned for treatment of bunions or other reasons. For example, metal plates, wires, and screws are routinely used to fixate bone fractures and osteotomies. These devices are used to reposition the bones or anchor the bones in the desired location and position so that proper bone alignment is maintained while the bones are healing. It is important to the successful outcome of the procedure that the materials used in bone fixation are compatible with human tissue while also sustaining the compressive forces helpful in promoting bone healing.

While the presently known methods for bone positioning and bone fixation are often successful, they are not without limitations and concerns. For example, the placement of screws and plates in the foot may compromise the bones and lead to additional fracturing in the future. Further, the screws, plates and wires may cause inflammation and swelling for some period of time after insertion into the bones and tenderness is not uncommon for prolonged periods of time. Accordingly, without improvements in the current drive system for positioning bones and providing proper bone fixation during orthopedic surgical procedures, the overall process and patient recovery rates from surgery will continue to be sub-optimal.

### BRIEF SUMMARY OF THE INVENTION

The various implementations of the present invention provide an improved apparatus for bone reduction and fixation. A flexible flat, substantially rectangular band comprises a wing or tab portion and a plurality of apertures formed in the body of the band. The band provides a mechanism for fixing bones in a desired position by encircling a first bone and a second bone. A first set of apertures provides for real time adjustment of the length of the band to allow for a wide variety of bone reduction applications. By selecting the appropriate length for the band, and inserting the wing portion in the appropriate slot, the band can be used for a wide range of applications. A second set of apertures formed in the band provide for bone and tissue contact, promoting healing of the body where the band is placed. A method for determining the proper positioning and placement of the wing portion during the bone reduction process is also disclosed.

### BRIEF DESCRIPTION OF THE FIGURES

The preferred embodiments of the present invention will hereinafter be described in conjunction with the appended drawings, wherein like designations denote like elements, and:

FIG. 1 is a perspective view of a bone band for bone fixation and reduction in accordance with a preferred exemplary embodiment of the present invention;

FIG. 2 is a top view of a bone band for bone fixation and reduction in accordance with a preferred exemplary embodiment of the present invention;

FIG. 3 is a bottom view of a bone band for bone fixation and reduction in accordance with a preferred exemplary embodiment of the present invention;

FIG. 4 is a left side view of a bone band for bone fixation and reduction in accordance with a preferred exemplary embodiment of the present invention;

FIG. 5 is a right side view of a bone band for bone fixation and reduction in accordance with a preferred exemplary embodiment of the present invention;

FIG. 6 is an end view of a bone band for bone fixation and reduction in accordance with a preferred exemplary embodiment of the present invention;

FIG. 7 is an end view of a bone band for bone fixation and reduction in accordance with a preferred exemplary embodiment of the present invention;

FIG. 8 is showing the placement of a bone band for bone fixation and reduction in accordance with a preferred exemplary embodiment of the present invention; and

FIG. 9 is a flow chart of a method of using a bone band for bone reduction and fixation in accordance with a preferred exemplary embodiment of the present invention.

### DETAILED DESCRIPTION OF THE INVENTION

The various implementations of the present invention provide an improved apparatus and method for bone reduction and fixation. A flexible, flat, substantially rectangular band comprises a wing portion and a plurality of apertures formed in the body of the band. The band provides a mechanism for fixing bones in a desired position by encircling a first bone and a second bone. A first set of apertures provides for real time adjustment of the length of the band to allow for a wide variety of bone reduction applications. By selecting the appropriate length for the band, and inserting the wing portion of the tab or head portion through the appropriate slot, the band can be used for a wide range of bone fixation and reduction applications. A second set of apertures formed in the band provide for bone and tissue contact, promoting healing of the area where the band is placed. A method for determining the proper positioning and placement of the bone band during the bone fixation and/or reduction process is also disclosed.

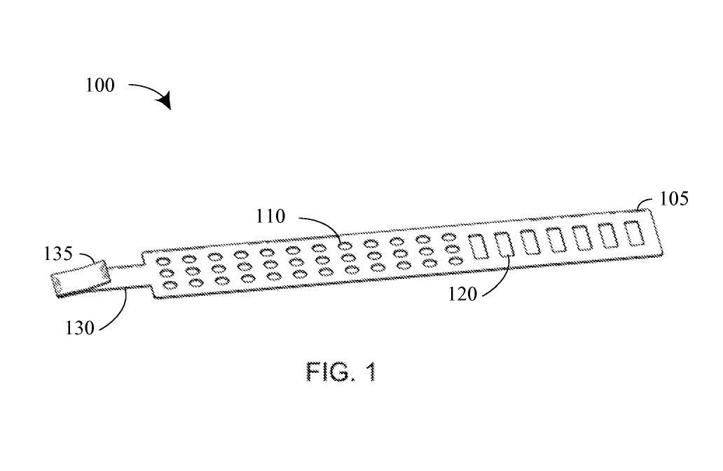

Referring now to FIG. 1, a bone band 100 for bone reduction/fixation in accordance with a preferred exemplary embodiment of the present invention is depicted. As shown in FIG. 1, bone band 100 is substantially flat and rectangular. Bone band 100 comprises a body portion 105 and a head or tab portion 130. Wing portion 135 is an integral of head or tab portion 130. A plurality of adjustment slots 120 are formed in body portion 105 and wing portion 135 may be inserted into adjustment slots 120 to adjust the length of bone band 100 as it is positioned around one or more bones. In the most preferred embodiments of the present invention, a plurality of contact apertures 110 are formed in the body of bone band 100 so as to prevent unnecessary isolation of the bone from the surrounding tissue. For promotion of proper healing, it is desirable to allow the tissue surrounding the bone to contact the bone as much as possible. Contact apertures 110 are sized and positioned so as to allow the bones to heal but are not so large and numerous as to compromise the structural integrity of bone band 100.

It is important to note the bone band 100 is most preferably manufactured from a metallic material that is safe for implantation into the human body. Those skilled in the art will recognize that many materials meet the requirements for suitable materials (e.g., biocompatible grade 316L stainless steel). Any biocompatible synthetic or natural material known to those skilled in the art may be employed. Additionally, the exact shape and size of bone band 100 will depend on the specific application (e.g., size and location of the bones, nature of the bone disorder or injury, etc.). In general, the shape of bone band 100 will rectangular and ribbon-like with smooth, round edges and corners. Bone band 100 should be thin enough to be flexible and bendable, allowing bone band 100 to be wrapped around bone surfaces while conforming generally to the bone surface.

Further, while adjustment apertures 120 are illustrated as being substantially rectangular in shape, other shapes may be used as appropriate. Similarly, contact apertures 110 are illustrated as being substantially circular in shape, other shapes may be suitable employed as well. No shape is excluded from consideration in various preferred embodiments of the present invention.

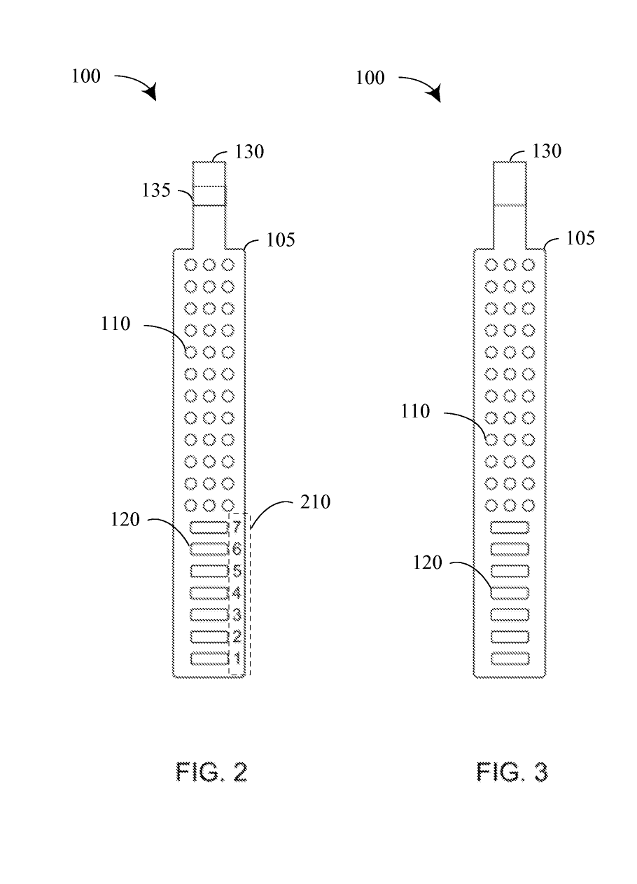

Referring now to FIG. 2, a top or front view of a bone band 100 for bone reduction/fixation in accordance with a preferred exemplary embodiment of the present invention is depicted. As shown in FIG. 2, body portion 105 of bone band 100 may comprise a series of measuring indicia 210 to assist the orthopedic surgeon in determining the appropriate adjustment slot 120 for proper bone positioning.

Referring now to FIG. 3, a bottom or back view of a bone band 100 for bone reduction/fixation in accordance with a preferred exemplary embodiment of the present invention is depicted.

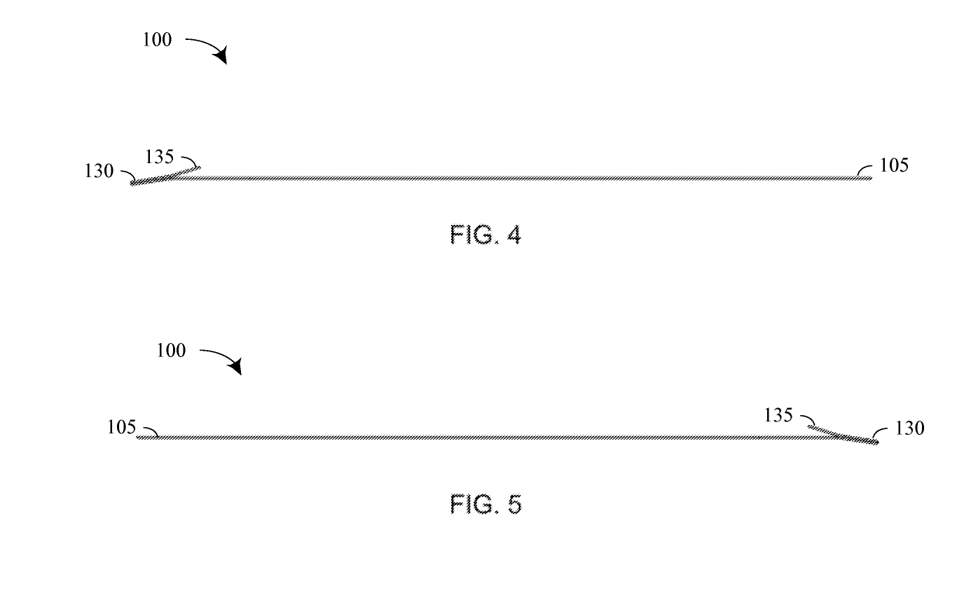

Referring now to FIG. 4, a side view of a bone band 100 for bone reduction/fixation in accordance with a preferred exemplary embodiment of the present invention is depicted. It should be noted that the flexible nature of the material used in manufacturing bone band 100 allows wing portion 135 to be “flexed” up and down to create the necessary angle for insertion into and through adjustment slots 120. Wing portion 135 can be selectively inserted into and removed from one or more adjustment apertures 120.

In the most preferred embodiments of the present invention, wing portion 135 is formed by doubling head or tab portion 130 back on itself to form the wing portion 135. At approximately a mid point of wing portion 135 a weld is made. From the weld point distally an extension head or tab portion is created. When wing portion 135 is engaged in slots 120, wing portion 135 cannot move beyond the slot bar further than the weld point. The extension head or tab portion when in the slots 120 prevents the Wing portion 135 from rotating out of the slots 120. In testing, this approach provides bone band 100 with as much as a 50% increase in pull testing to failure.

Referring now to FIG. 5, a side view of a bone band 100 for bone reduction/fixation in accordance with a preferred exemplary embodiment of the present invention is depicted.

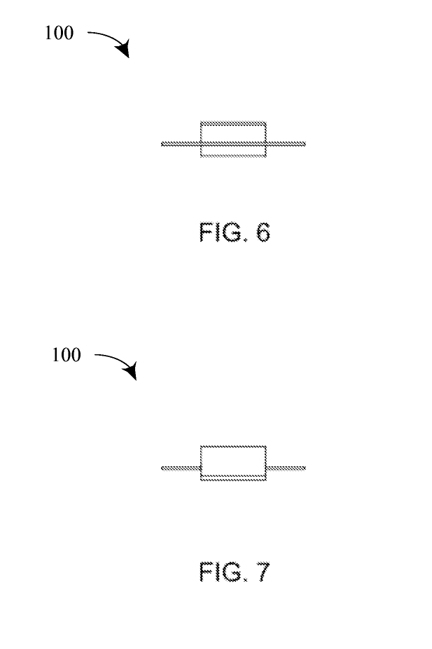

Referring now to FIG. 6, an end view of a bone band 100 for bone reduction/fixation in accordance with a preferred exemplary embodiment of the present invention is depicted.

Referring now to FIG. 7, an end view of a bone band 100 for bone reduction/fixation in accordance with a preferred exemplary embodiment of the present invention is depicted.

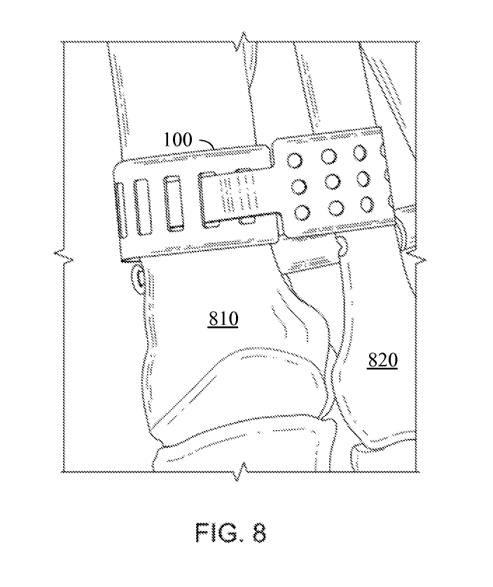

Referring now to FIG. 8, bone band 100 is shown in an application for reduction/fixation of a bone in accordance with a preferred exemplary embodiment of the present invention is depicted. When bone band 100 is used to provide bone fixation, it will be positioned so as to encircle at least two bones or bone segments. One of the bones or bone segments will be used as an “anchor” to hold the second bone or bone segment in the desired position. In at least some preferred embodiments of the present invention, the two bone segments will be in the fracture of a single bone and bone band 100 will be utilized to help pull the bone segments back into an aligned position. As shown in FIG. 8, bone band 100 is positioned around a first bone 810 and a second bone 820. This allows a bone to be positioned and stabilized relative to the position of the neighboring bone.

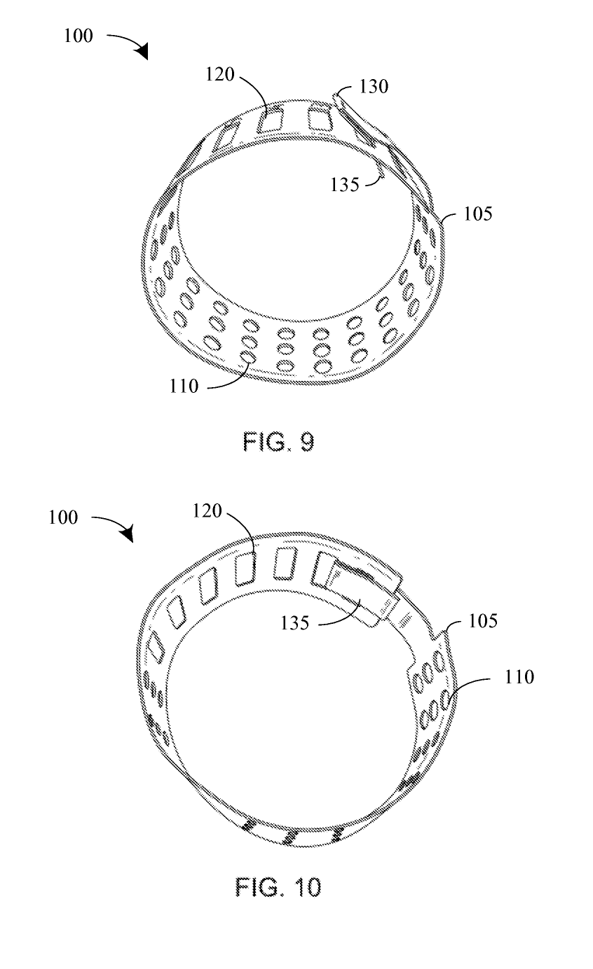

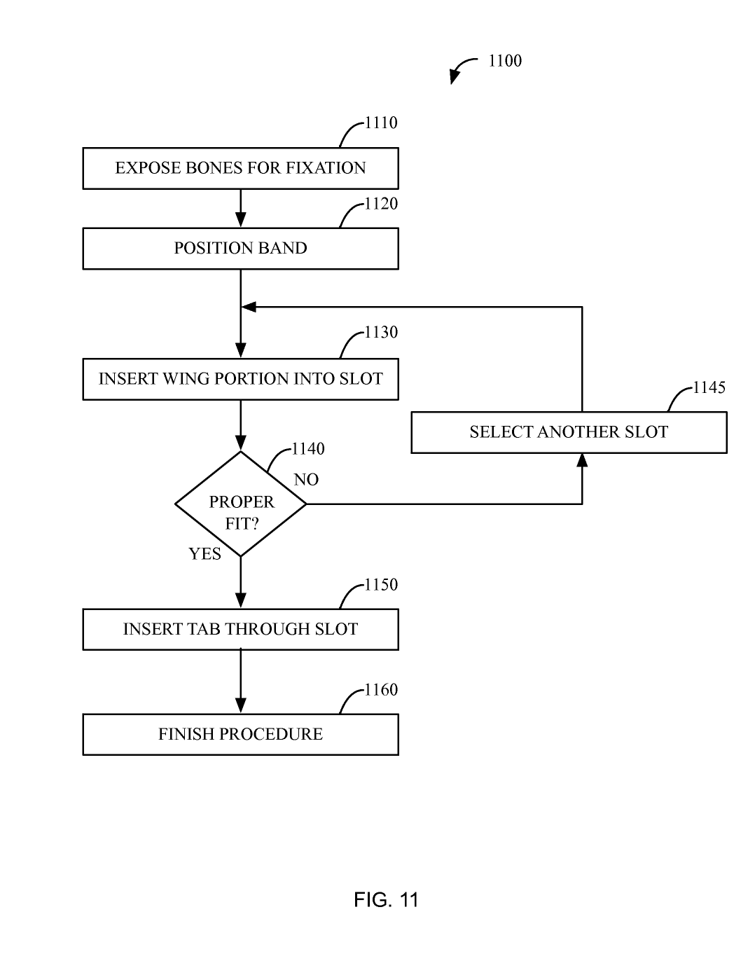

Referring now to FIG. 9, the use of bone band 100 for bone fixation is depicted. As shown in FIG. 9, bone band 100 has been formed into a substantially rectangular shape. This illustrates the use of bone band 100 to encircle the bones or bone fragments (not shown this FIG.). Additionally, it should be noted that wing portion 135 has been inserted through an adjustment aperture 120 as a preliminary measuring step. Wing portion 135 is positioned on the interior of the circular shape formed by body portion 105 but the remainder of head or tab portion 130 remains on the exterior of the circular shape formed by body portion 105 of bone band 100. This allows the orthopedic surgeon to “test fit” the positioning and sizing of bone band 100. If necessary, wing portion can be removed from the preliminarily selected adjustment aperture 120 and relocated to a more appropriate adjustment aperture 120. By selectively inserting and removing wing portion 135 through one or more adjustment apertures 120, the orthopedic surgeon can select the most appropriate location and adjust the relative length of bone band 100 to achieve the desired results.

Referring now to FIG. 10, wing portion 135 and head or tab portion 130 have both been inserted completely through an adjustment aperture 120. In this fashion, head or tab portion 130 is now positioned on the interior of the circular shape formed by body portion 105. This effectively “locks” the size and circumference of circular shape formed by body portion 105 and will provide for a stabilized bone structure.

Referring now to FIG. 11, a flow chart for a method 1100 of bone fixation and reduction using a bone band in accordance with a preferred exemplary embodiment of the present invention is depicted. As shown in FIG. 11, the orthopedic surgeon will begin the procedure by exposing the bone or bones that need to be reduced and/or fixed in position (step 1110). The orthopedic surgeon will position the band around the bone(s) in an appropriate position so as to most effectively position the bones for proper healing (step 1120). Once the orthopedic surgeon believes the bone band is properly positioned, the wing portion of the tab can be inserted into a positioning slot to verify the length of the band will accomplish the proper bone positioning (step 1130).

If the length and fit of the bone band as initially selected by the orthopedic surgeon do not provide the desired reduction (step 1140=“NO”), then the orthopedic surgeon will reposition the bone band and select another positioning slot to obtain a more appropriate result (step 1145). Once the new slot has been selected, the orthopedic surgeon will once again insert the wing portion of the tab into the newly selected slot (step 1130). This adjustment process will continue until the orthopedic surgeon has achieved the desired result.

Once the selected slot provides a band of the proper length and fit (step 1140=“YES”), then the entire locking tab will be inserted through the positioning slot (step 1150), thereby locking the band into the desired position. The orthopedic surgeon can then take whatever additional steps may be necessary to complete the procedure (step 1160) such as cleaning the tissue around the band and suturing any incisions, etc. It should be noted that this entire process might be repeated as necessary for additional bones.

From the foregoing description, it should be appreciated that the bone fixation device and methods disclosed herein presents significant benefits that would be apparent to one skilled in the art. Furthermore, while multiple embodiments have been presented in the foregoing description, it should be appreciated that a vast number of variations in the embodiments exist. Lastly, it should be appreciated that these embodiments are preferred exemplary embodiments only and are not intended to limit the scope, applicability, or configuration of the invention in any way. Rather, the foregoing detailed description provides those skilled in the art with a convenient road map for implementing a preferred exemplary embodiment of the invention, it being understood that various changes may be made in the function and arrangement of elements described in the exemplary preferred embodiment without departing from the spirit and scope of the invention as set forth in the appended claims.

1. An apparatus comprising:
a substantially flat, flexible, rectangular body portion,
a plurality of adjustment apertures formed in the body portion;
a plurality of contact apertures formed in the body portion; and
a head portion;
a wing portion, the wing portion being selectively inserted into and removed from at least one of the plurality of adjustment apertures and the head portion being inserted fully through the at least one of the plurality of adjustment apertures.

2. The apparatus of claim 1 wherein the plurality of adjustment apertures comprise a plurality of rectangular apertures.

3. The apparatus of claim 1 wherein the plurality of contact apertures comprise a plurality of circular apertures.

4. The apparatus of claim 1 wherein the plurality of adjustment apertures comprise a plurality of rectangular apertures and wherein the plurality of contact apertures comprise a plurality of circular apertures.

5. The apparatus of claim 1 further comprising at least two bone segments wherein the body portion encircles the at least two bone segments.

6. The apparatus of claim 1 wherein the wing portion is partially inserted into the at least one adjustment aperture prior to the head portion being inserted through the at least one of the plurality of adjustment apertures.

7. The apparatus of claim 1 wherein the apparatus is manufactured from a biocompatible stainless steel material.

8. The apparatus of claim 1 wherein the plurality of adjustment apertures comprise a plurality of rectangular apertures and wherein the plurality of contact apertures comprise a plurality of circular apertures and wherein the apparatus is manufactured from a biocompatible stainless steel material.

9. The apparatus of claim 1 further comprising a plurality of indicia on the body portion, the plurality of indicia being positioned adjacent to the plurality of adjustment apertures.

10. A method comprising the steps of:
exposing at least a first bone segment;
exposing at least a second bone segment; and
encircling the at least a first bone segment and the at least a second bone segment with a bond band.

11. The method of claim 10 wherein the bone band comprises:
a substantially flat, flexible, rectangular body portion,
a plurality of adjustment apertures formed in the body portion;
a plurality of contact apertures formed in the body portion; and
a head portion;
a wing portion, the wing portion being selectively inserted into and removed from at least one of the plurality of adjustment apertures and the head portion being inserted fully through the at least one of the plurality of adjustment apertures.

12. The method of claim 11 wherein the plurality of adjustment apertures comprise a plurality of rectangular apertures and wherein the plurality of contact apertures comprise a plurality of circular apertures and wherein the apparatus is manufactured from a biocompatible stainless steel material.

13. The method of claim 10 wherein the bone band is manufactured from a biocompatible stainless steel material.

14. The method of claim 10 wherein the step of encircling the at least a first bone segment and the at least a second bone segment with a bond band comprises the steps of:
partially inserting a wing portion of the bone band into a first adjustment aperture;
removing the wing portion of the bone band from the first adjustment aperture; and
inserting the wing portion of the bone band fully through a second adjustment aperture.

15. The method of claim 10 wherein the bone band comprises a plurality of substantially rectangular adjustment apertures.

16. The method of claim 10 wherein the bone band comprises a plurality of substantially circular contact apertures.

17. The method of claim 10 wherein the bone band comprises:
a plurality of substantially rectangular adjustment apertures; and
a plurality of substantially circular contact apertures.
